(19) United States
(12) Patent Application Publication
(10) Pub. No.: US 2026/0199039 A1
(43) Pub. Date: Jul. 16, 2026

(54) SURGICAL ROBOTIC SYSTEM AND METHOD FOR DISPLAYING INCREASED LATENCY

(71) Applicant: Covidien LP, Mansfield, MA (US)

(72) Inventor: Scott Hopkinson, Concord, MA (US)

(21) Appl. No.: 19/138,240

(22) PCT Filed: Dec. 11, 2023
(86) PCT No.: PCT/IB2023/062504

§ 371 Date: Jun. 12, 2025

Related U.S. Application Data
(60) Provisional application No. 63/433,502, filed on Dec. 19, 2022.

(51) Int. Cl. A61B 34/37; A61B 34/20; G16H 40/67

(52) U.S. Cl. CPC A61B 34/37; G16H 40/67; A61B 2034/2055

(57) ABSTRACT

A surgical robotic system includes a surgical robotic arm having an instrument and an instrument drive unit configured to actuate the instrument, and a surgeon console configured to receive user input to control at least one of the surgical robotic arm or the instrument. The system also includes a camera configured to capture a video feed and a video processing device configured to receive the video feed, calculate a latency of the video feed, and display the latency of the video feed.

This application claims the benefit of U.S. Provisional Ser. No. 63/433,502, filed Dec. 19, 2022, the entire content of which is incorporated herein by reference.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Laparoscopic and robotic surgery rely on real-time visualization. The surgical site is shown on a display within a short latency of up to about 80 milliseconds (ms) between the time the surgical site is captured by a camera and when it is output on a display. This allows the surgeon to view the surgical site in nearly real-time while performing surgical procedures such that there is little to no noticeable delay between surgeon's inputs and resulting movement of instruments at the surgical site. Occasionally latency can increase due to visual enhancements, e.g., artificial intelligence (AI) overlays, near infrared imaging, and other techniques in addition to white light laparoscopic imaging. Increased latency may be unacceptable due to the delay between user input (e.g., moving an instrument) and the movement of the instrument being displayed on a screen. Thus, there is a need to alert the user of increased latency.

### SUMMARY

Latency in surgical visualization may be caused by a variety of factors including, but not limited to, video processing delays, transmission delays, AI processing, near infrared (NIR) imaging, and the like. Thus, latency may fluctuate during use of the visualization system depending on various processing enhancements.

The present disclosure provides for a surgical robotic system and method for alerting a user of increased latency. The system also outputs a query asking whether the user would like to continue operation of the system when high latency is encountered, thus giving the user opportunity to stop (e.g., temporarily) or continue the operation despite the latency. After dropping below a preset threshold, the user may be prompted to resume the operation. The latency threshold may be automatically preset in the system or adjustable by the user. In embodiments, there may be different types of thresholds depending on the type of visualization, such as white light imaging, NIR imaging using a dual sensor (e.g., white light and NIR sensors), etc. The white light imaging may have a latency threshold of about 145 ms, while NIR enhanced imaging may have a latency threshold of about 165 ms to account for processing NIR light images in addition to white light images. The user may adjust these thresholds based on their tolerance for latency.

According to one embodiment of the present disclosure, a surgical robotic system is disclosed. The surgical robotic system includes: a surgical robotic arm having an instrument and an instrument drive unit configured to actuate the instrument; and a surgeon console configured to receive user input to control at least one of the surgical robotic arm or the instrument. The system also includes a camera configured to capture a video feed and a video processing device configured to receive the video feed, calculate a latency of the video feed, and display the calculated latency of the video feed.

Implementations of the above embodiment may include one or more of the following features. According to one aspect of the above embodiment, the video processing device may be further configured to compare the latency of the video feed to at least one threshold. The video processing device may be further configured to output an indication in response to the latency of the video feed exceeding the at least one threshold. The indication may include at least one of a color-coded latency number, a color-coded frame, or a color-coded tint of the video feed. The video processing device may be further configured to output a prompt querying whether to continue or pause operation in response to the latency of the video feed exceeding the at least one threshold. The camera may be configured to capture white light and near infrared (NIR) light, and the video processing device may be configured to perform at least one of white light imaging or combined white and NIR light imaging. The at least one threshold may be a user-adjustable value. The at least one threshold may include a first threshold for white light imaging and a second threshold for combined white and NIR light imaging. The first threshold may be lower than the second threshold. The first threshold may be 145 milliseconds. The second threshold may be 165 milliseconds.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of the surgical robotic system of FIG. 1 positioned about a surgical table according to an embodiment of the present disclosure;

FIG. 6 is a view of a screen of a surgeon console displaying advisories of increased latency according to an embodiment of the present disclosure; and

FIG. 7 is a flow chart illustrating a method for displaying an advisory of increased latency according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands and to generate torque commands for activating one or more actuators of the robotic arm, which would, in turn, move the robotic arm in response to the movement command.

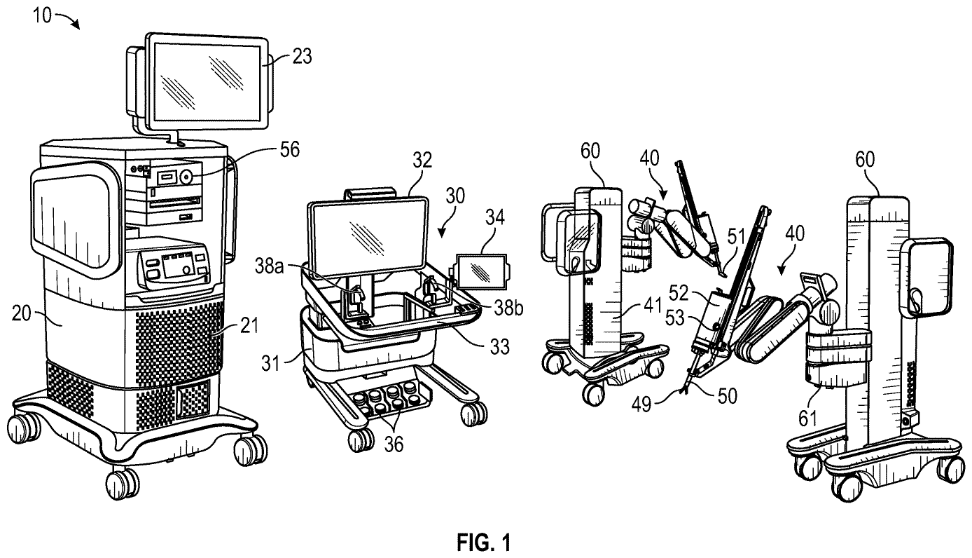

With reference to FIG. 1, a surgical robotic system 10 includes a control tower 20, which is connected to all of the components of the surgical robotic system 10 including a surgeon console 30 and one or more mobile carts 60. Each of the mobile carts 60 includes a robotic arm 40 having a surgical instrument 50 removably coupled thereto. The robotic arms 40 also couple to the mobile carts 60. The robotic system 10 may include any number of mobile carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include a laparoscopic camera 51 configured to capture video of the surgical site. The laparoscopic camera 51 may be a stereoscopic endoscopic camera configured to capture two side-by-side (i.e., left and right) images of the surgical site to produce a video stream of the surgical scene. The laparoscopic camera 51 is coupled to an image processing device 56, which may be disposed within the control tower 20. The image processing device 56 may be any computing device configured to receive the video feed from the laparoscopic camera 51 and output the processed video stream.

The surgeon console 30 includes a first screen 32, which displays a video feed of the surgical site provided by camera 51 of the surgical instrument 50 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of user interface devices, such as foot pedals 36 and a pair of hand controllers 38a and 38b which are used by a user to remotely control robotic arms 40. The surgeon console further includes an armrest 33 used to support clinician's arms while operating the hand controllers 38a and 38b.

The control tower 20 includes a screen 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the hand controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DC). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

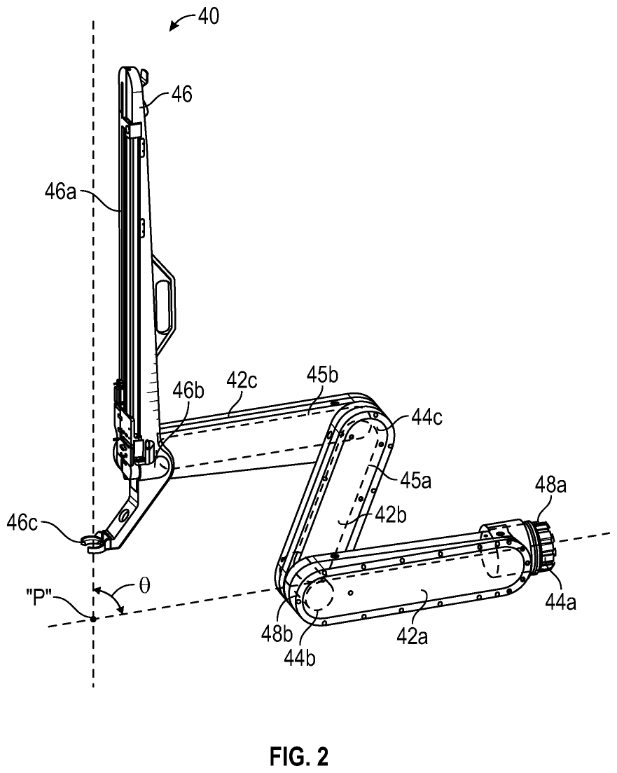

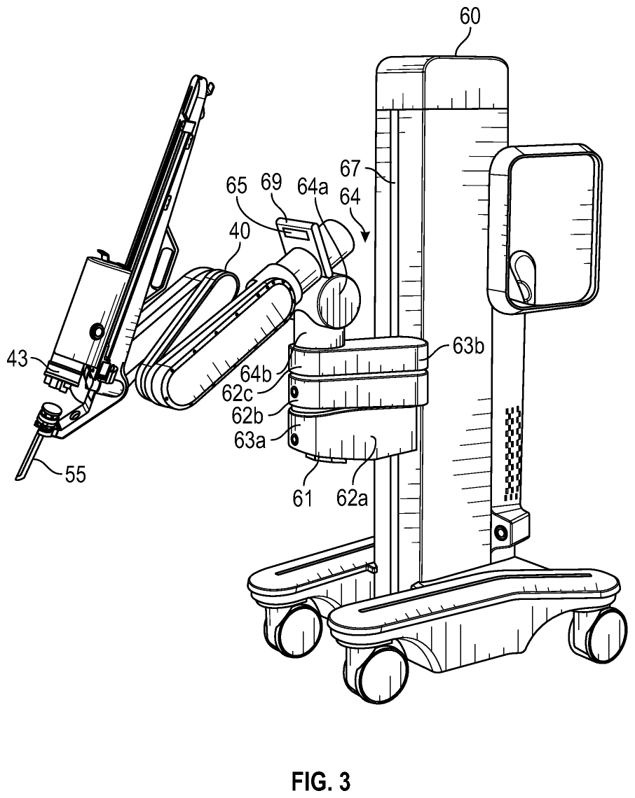

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a screen 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

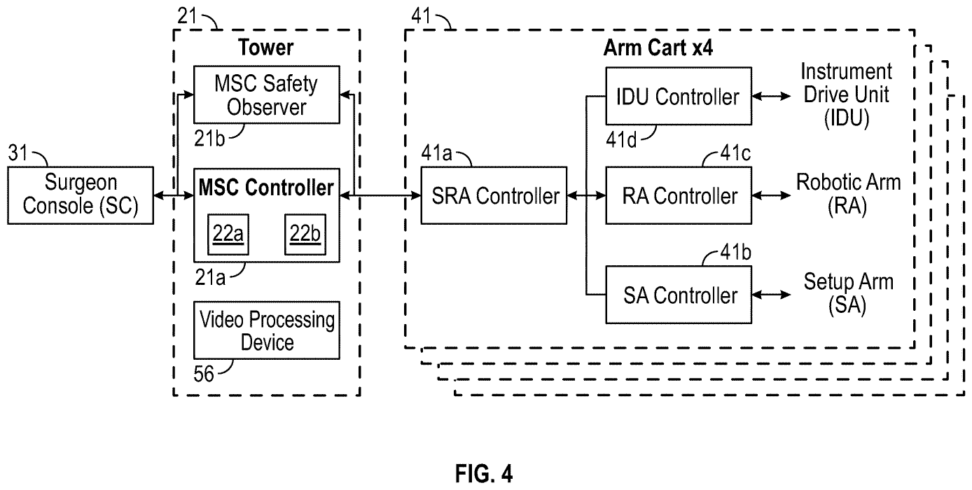

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the hand controller 38a is then scaled by a scaling function executed by the controller 21a. In embodiments, the coordinate position may be scaled down and the orientation may be scaled up by the scaling function. In addition, the controller 21a may also execute a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

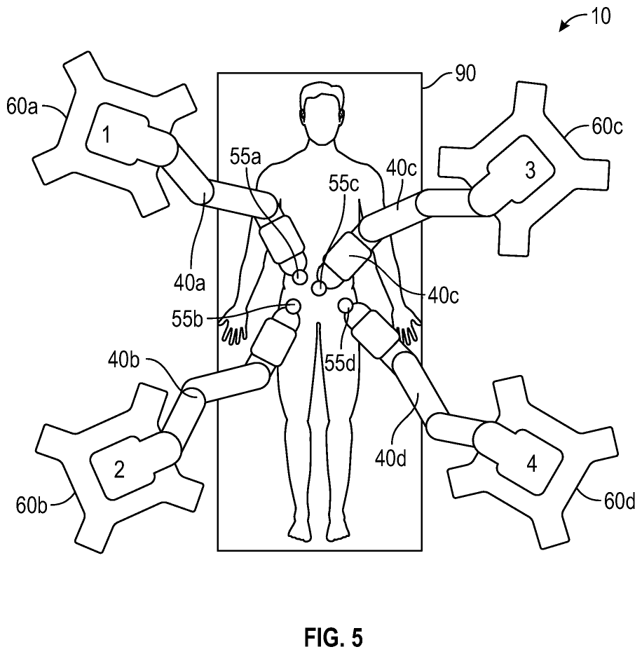

With reference to FIG. 5, the surgical robotic system 10 is setup around a surgical table 90. The system 10 includes mobile carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the laparoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52. The SIM 43 is also configured to secure a sterile drape (not shown) to the IDU 52.

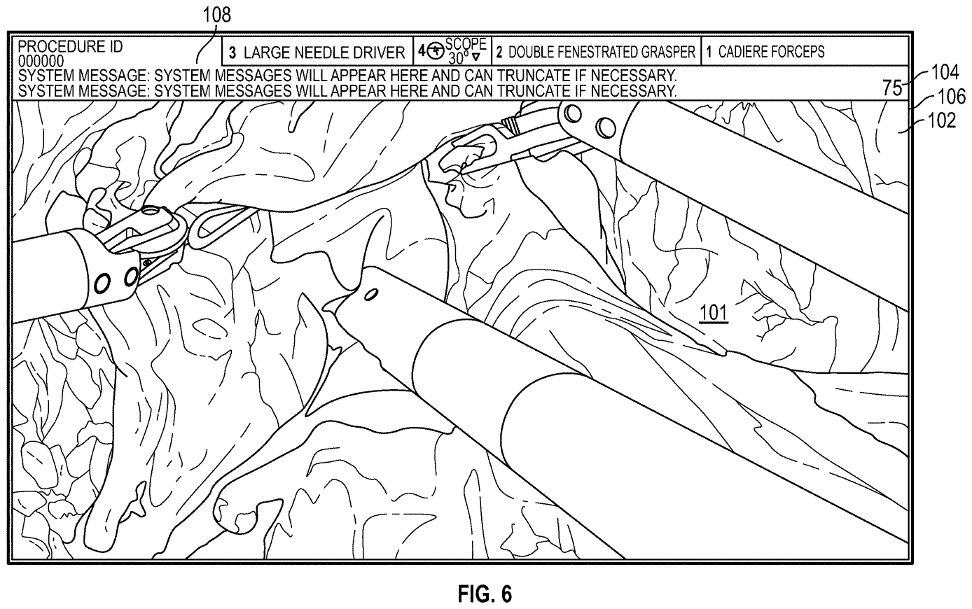

With reference to FIG. 6, the first screen 32 of the surgeon console 30 includes a GUI 101 providing a video feed 102 of the camera 51. The video feed 102 is within a field of view of the camera 51 and may show the surgical site, the instruments 50, etc. The video processing device 56 is configured to output the GUI 101, which may be displayed on any of the screens of the system 10, e.g., the second screen 34 of the surgeon console 30, the screen 23 of the control tower 20, etc.

The GUI 101 may be implemented in any laparoscopic visualization system, not just the surgical robotic system 10 of the present disclosure. Thus, any surgical operation using manual, powered, or robotic surgical instruments may be modified using the GUI 101 by incorporating the latency tracking and prompts provided by the GUI 101. Thus, the video processing device 56 along with the camera 51, which may be any surgical camera, e.g., open, endoscopic, capsule, laparoscopic, etc. may be used in any surgical setting utilizing real-time visualization.

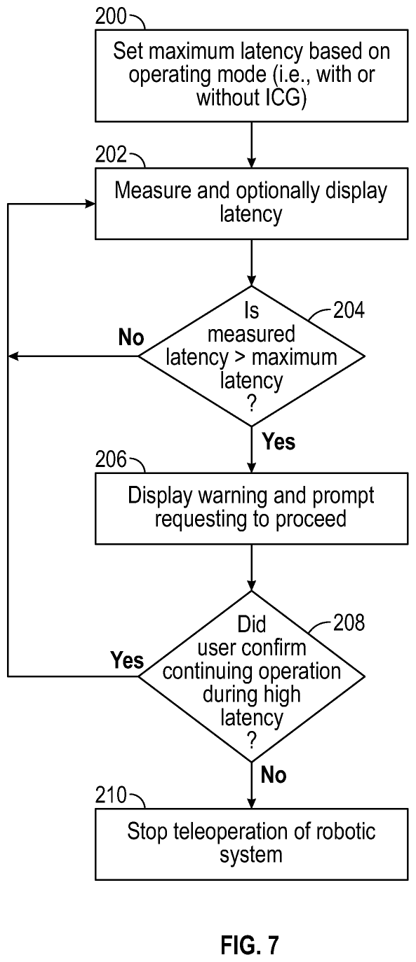

With reference to FIG. 7, a method for tracking and displaying latency in a surgical visualization system includes setting a maximum latency for the visualization system, which includes the video processing device 56 and the camera 51. The method may be embodied as software instructions stored in non-transitory medium (e.g., memory) of the video processing device 56 and executable by one or more processors (e.g., FPGA, CPU, GPU, etc.) of the video processing device 56.

The camera 51 may be a dual sensor camera capable of imaging white light and NIR imaging using various contrast agents. With intraoperative usage of fluorophores from a fluorescent dye, such as indocyanine green (ICG), the imaging system enables real-time visual assessment and of blood vessels, lymph nodes, lymphatic flow, biliary ducts, and other tissues during surgical procedures. The video processing device 56 is configured to combine the white light and IR images from the camera 51 by displaying reflected NIR light as a visible color (e.g., green, blue, etc.) on the video feed 102.

At step 200, one or more latency thresholds are set, which may be done by the user prior to or during the procedure. In embodiments, the latency thresholds may be set by the system 10, i.e., as a default parameter. The video processing device 56 may include a threshold for different types of imaging being performed. Thus, for white light imaging, the video processing device 56 may have a first threshold, which may be from about 100 ms to about 150 ms, and in embodiments may be about 145 ms. For NIR imaging, due to increased latency for combing white and NIR images, the video processing device 56 may have a second threshold, which may be from about 120 ms to about 180 ms, and in embodiments may be about 165 ms. The thresholds may also be dynamically adjusted based on which imaging mode is being used by the video processing device 56. Thus, as different modes are activated or deactivated, corresponding thresholds are selected by the video processing device 56.

At step 202, the video processing device 56 measures latency of the video feed. This may be done continuously or periodically at any suitable frequency (60 Hz) during the processing of the video feed 102. Any suitable technique for measuring latency of the video feed may be used, e.g., time stamps, round trip time, etc.

The video processing device 56 compares the measured latency to the threshold latency at step 204, as previously set at step 200. This may also be done continuously or periodically at any suitable frequency (60 Hz) during the processing of the video feed 102. The measured latency may also be displayed on the GUI 101 as a number 104. The number 104 may be color-coded to indicate the current latency range; thus, low latency may be colored using green, middle latency as yellow, and high latency, i.e., above or approaching the threshold, may be red. Additional indication of high latency may be done by using a color-coded frame 106 around the video feed 102. In embodiments, the video feed 102 may be tinted with the same or similar color codes to indicate an increase in latency. Thus, low latency may be denoted without tint and high latency may be denoted by red tinting of the video feed 102. In addition to the number 104 and color coding of the frame 106 and/or the video feed 102, latency numbers and status may also be displayed in a system message area 108 along with other status messages as they appear.

If the latency is below the threshold, the method returns to step 202 to measure latency and compare the latency at step 204. However, if the latency is above the threshold, at step 206, the video processing device 56 displays a warning regarding the latency exceeding the threshold. This may be done through the message area 108.

In embodiments, a prompt may be displayed over the video feed 102 notifying the user of the high latency. The prompt may also request an input from the user at step 208. The input may be a response to a question asking the user whether to continue the current operation or to stop or pause the procedure. If the user answers yes, then the system 10 continues operation and the video processing device 56 returns to step 202 to measure latency and compare the latency at step 204. If the user answers no, the system 10 may stop or pause operation at step 210. This may be done for a period of time until the latency of the video feed lowers below the threshold and/or for a predetermined period of time, which may be from about 10 seconds to about 5 minutes. In embodiments, the system 10 may pause or stop indefinitely until the user actively resumes operation of the system 10 through the surgeon console 30.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a surgical robotic arm including an instrument and an instrument drive unit configured to actuate the instrument;
a surgeon console configured to receive user input to control at least one of the surgical robotic arm or the instrument;
a camera configured to capture a video feed; and
a video processing device configured to:
receive the video feed;
calculate a latency of the video feed; and
display the latency of the video feed.

2. The surgical robotic system according to claim 1, wherein the video processing device is further configured to compare the latency of the video feed to at least one threshold.

3. The surgical robotic system according to claim 2, wherein the video processing device is further configured to output an indication in response to the latency of the video feed exceeding the at least one threshold.

4. The surgical robotic system according to claim 3, wherein the indication includes at least one of a color-coded latency number, a color-coded frame, or a color-coded tint of the video feed.

5. The surgical robotic system according to claim 3, wherein the video processing device is further configured to output a prompt querying whether to continue or pause operation of the surgical robotic system in response to the latency exceeding the at least one threshold.

6. The surgical robotic system according to claim 2, wherein the camera is configured to capture white light and near infrared (NIR) light, and the video processing device is configured to perform at least one of white light imaging or combined white and NIR light imaging.

7. The surgical robotic system according to claim 6, wherein the at least one threshold is a user-adjustable value.

8. The surgical robotic system according to claim 6, wherein the at least one threshold includes a first threshold for white light imaging and a second threshold for combined white and NIR light imaging.

9. The surgical robotic system according to claim 8, wherein the first threshold is lower than the second threshold.

10. The surgical robotic system according to claim 8, wherein the first threshold is 145 milliseconds.

11. The surgical robotic system according to claim 8, wherein the second threshold is 165 milliseconds.
